(19) United States
(12) Patent Application Publication
(10) Pub. No.: US 2018/0344721 A1
(43) Pub. Date: Dec. 6, 2018

(54) Medicinal Treatment of Chronic Inflammatory Dermal Disorders

(71) Applicant: Bridge Pharma, Inc., Sarasota, FL (US)

(72) Inventors: A.K. Gunnar Aberg, Sarasota, FL (US); Vincent B. Ciofalo, Branford, CT (US)

(21) Appl. No.: 16/100,424

(22) Filed: Aug. 10, 2018

Related U.S. Application Data
(63) Continuation of application No. 13/744,807, filed on Jan. 18, 2013, now abandoned.
(60) Provisional application No. 61/720,058, filed on Oct. 30, 2012.

(51) Int. Cl. A61K 31/4535; A61K 31/56; A61K 45/06

(52) U.S. Cl. CPC A61K 31/4535; A61K 31/56; A61K 2300/00; A61K 45/06

(57) ABSTRACT

The methods disclosed herein relate to the treatment of chronic inflammatory dermal disorder in humans, by administering a therapeutically effective amount of RS-norketotifen.

### CROSS-REFERENCE TO RELATED APPLICATIONS

This application is a continuation of U.S. Application Serial No. 13/744,807 filed on Jan. 18, 2013, which claims priority of U.S. Provisional Application Ser. No. 61/720,058, filed on Oct. 30, 2012, the disclosure of which are hereby incorporated by reference.

### TECHNICAL FIELD

The embodiments disclosed herein relate to methods of treatment of inflammatory and allergic diseases while avoiding side effects that are commonly associated with anti-inflammatory antihistamines.

### BACKGROUND

Ketotifen (Zaditen®, Zaditor®, Sandoz, Novartis) is a Generation-1 antihistamine that is mainly used for the treatment of allergic rhinitis. Ketotifen may be the most sedating of all marketed antihistamines, and the unusually severe sedative side effects of ketotifen have limited the therapeutic usefulness of the drug. In the USA, ketotifen is only used as eye drops (Zaditor®, Novartis) to alleviate the symptoms of allergic conjunctivitis in humans. The eye drop formulation does not cause sedation due to the extremely low systemic concentrations of the drug after local administration to the eyes.

Norketotifen, an active metabolite of ketotifen, is an achiral molecule, but has two atropisomers, S-norketotifen and R-norketotifen, as has previously been described in U.S. Pat. Nos. 7,226,934 and 7,557,128. As explained in U.S. Pat. Nos. 7,226,934 and 7,557,128, norketotifen also has a significant sedation effect when studied in the art-accepted mouse model of sedation, and further, the sedative effects were attributed to the R-isomer. It was thus proposed that only the S-isomer could be administered without significant sedation effects.

What is needed are methods for oral treatment of atopic inflammatory disorders with low-toxic oral medication without causing sedation or the much feared side effects of corticosteroids and immunosuppressant drugs.

### SUMMARY

In one aspect, a method for treating atopic inflammatory disorders in a human patient in need thereof comprises orally administering to the human patient a therapeutically effective amount of RS-norketotifen or a pharmaceutically acceptable salt thereof, wherein the therapeutically effective amount of RS-norketotifen or a pharmaceutically acceptable salt thereof does not produce sedative side effects in the human patient. In further embodiments, norketotifen does not cause the side effects of long-term administration of corticosteroids or the side effects of immunosuppressant drugs.

In another aspect, a method of reducing sedative side effects in the treatment of atopic inflammatory disorders in a human patient in need thereof comprises orally administering to the human patient in need a therapeutically effective amount of norketotifen or a pharmaceutically acceptable salt thereof that does not produce sedative side effects upon administration to the human patient.

In a further aspect, a method of treating an atopic inflammatory disorder in a human patient comprises determining whether said patient is susceptible to adverse sedative effects of compounds with antihistaminic activity, and if said determination is positive, administering to said patient in need thereof an oral formulation comprising a therapeutically effective amount of norketotifen or a pharmaceutically acceptable salt of norketotifen.

### DETAILED DESCRIPTION

The methods disclosed herein relate to the treatment of atopic inflammatory disorders including dermal disorders, such as for example eczema, atopic dermatitis, urticaria and psoriasis; pulmonary disorders, such as asthma, chronic obstructive pulmonary disease (COPD), cough, bronchial hyperreactivity, and bronchitis; and gastrointestinal disorders, including gastric irritation, gastric allergic disorders and gastric inflammatory disorders in patients. The methods include administering a compound with combined anti-inflammatory and anti-allergic activities.

It has previously been found and described that both ketotifen and norketotifen express sedative activity and were therefore not considered to be useful as medications for the treatment of chronic disorders, such as for example, chronic dermal disorders, chronic pulmonary disorders and chronic gastrointestinal disorders. Sedation was determined using a mouse model that has previously been used successfully in the development of non-sedating antihistamines, such as loratadine (Claritin®, Schering) and desloratadine (Clarinex®, Schering). The mouse model identified known sedating (Generation-1) and non-sedating (Generation-2) antihistaminic compounds with complete accuracy and the model also clearly demonstrated sedative effects of norketotifen (Table 5). It was therefore believed that the mouse model had relevance for predicting sedative side effects also of norketotifen that has potent antihistaminic activities.

It has now surprisingly been found that racemic norketotifen is completely free from sedative effects when tested in humans, even after oral administration of high doses of the compound, such as 10 mg, twice daily.

The active compound described herein is racemic norketotifen, herein also called RS-norketotifen, nor-ketotifen or just norketotifen. RS-norketotifen is an achiral molecule that has two isomers, S-norketotifen and R-norketotifen, which are atropisomers, as has previously been described in U.S. Pat. Nos. 7,226,934 and 7,557,128.

Chemically, the benzocycloheptathiophene compound RS-norketotifen is called (RS)-4-(4-piperidylidene)-9,10-dihydro-4H-benzo-(4,5)-cyclohepta (1,2-b) thiophene-10-one. The prefix (RS) can optionally be excluded.

Norketotifen is a metabolite of ketotifen (4-(1-methyl-4-piperidyline)-4H-benzo(4,5) cyclohepta-(1,2-b) thiophene-10-one). Ketotifen (Zaditen®, Zaditor®, Sandoz, Novartis) is a Generation-1 antihistamine that is mainly used for the treatment of allergic rhinitis. Ketotifen may be the most sedating of all marketed antihistamines and the unusually severe sedative side effects of ketotifen are dose limiting to 1 mg, bid.

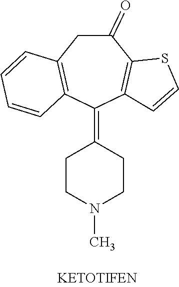 [SMILES: CN1CCC(=C2c3ccccc3CC(=O)c3sccc32)CC1]

The metabolite norketotifen is formed by demethylation of ketotifen in the liver of mammals

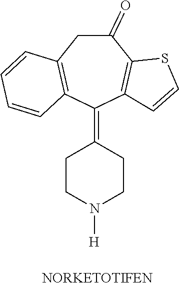 [SMILES: O=C1Cc2ccccc2C(=C2CCNCC2)c2ccsc21]

Norketotifen can be made from methods known in the art, as described in U.S. Pat. No. 3,682,930, the disclosure of which is hereby incorporated by reference for its teaching of the synthesis of norketotifen.

Except for U.S. Pat. Nos. 7,226,934 and 7,557,128, no publications are known that describe the pharmacodynamic activities of norketotifen. U.S. Patent Publications 2010/0105734 and 2010/0130550 describe the effects of norketotifen when used as eye drops for ocular conjunctivitis and xerophthalmia in humans.

The embodiments disclosed herein provide for the administration of the racemic form of norketotifen and the pharmaceutically acceptable acid addition salts thereof to patients suffering from inflammatory dermal disorders, inflammatory pulmonary disorders or inflammatory gastrointestinal disorders. Norketotifen is ideally suited for the treatment of allergic and inflammatory dermal, pulmonary diseases and gastrointestinal disorders, since this compound, has potent anti-inflammatory and antihistaminic effects and low systemic toxicity, and has now, surprisingly, been found to be completely free from sedative side effects in humans. In one embodiment, administration is oral administration. In another embodiment, administration is by inhalation.

Allergic and or inflammatory disorders include dermal disorders such as for example eczema, atopic dermatitis, urticaria and psoriasis. Pulmonary disorders include for example asthma, COPD, cough, bronchial hyperreactivity, and bronchitis, and gastrointestinal disorders including gastric irritation, gastric allergic disorders, and gastric inflammatory disorders. Examples of gastric inflammatory disorders are for example various eosinophilic gastrointestinal disorders, such as for example eosinophilic esophagitis, eosinophilic gastroenteritis and other forms of intestinal eosinophilia.

The term atopic inflammatory disorders is used herein to denote dermal disorders, such as for example eczema, atopic dermatitis, urticaria and psoriasis; pulmonary disorders, such as for example asthma, COPD, bronchitis, bronchial hyperactivity and cough; and gastrointestinal disorders, such as for example, gastric irritation, gastric allergic disorders and gastric inflammatory disorders.

Norketotifen is particularly useful in the treatment of human patients who are susceptible to sedation upon administration of drugs with antihistaminic activity. Sedation is a common side effect of antihistamines such as diphenhydramine, which is a sedating Generation-1 antihistamine without anti-inflammatory activity used mainly for the treatment of allergic rhinitis. Ketotifen is also known to cause sedation. For some patients, the sedative side effects can outweigh the benefits of such medication, particularly in the treatment of chronic, non-life-threatening conditions such as dermal disorders, such as for example atopic dermatitis, and pulmonary disorders, such as for example asthma. While allergic rhinitis is a highly seasonal condition, dermal and pulmonary disorders most often require year-round treatment over multiple years. Sedation over long periods of time is not acceptable. Further, while non-sedating antihistamines are available, these drugs do not have the anti-inflammatory activity needed for the treatment of inflammatory disorders, such as for example atopic dermatitis and asthma. Because of the previous belief that norketotifen has similar sedative side effects as ketotifen, one would not have used this compound to treat chronic allergic/inflammatory disorders in patients susceptible to sedative side effects.

Those skilled in the art know how to determine if a patient is susceptible to sedation from sedative (Generation-1) compounds. For example, the oral administration of a single daily clinical dose of ketotifen will cause sedation in individuals susceptible to sedative side effects, but the dose of ketotifen will not cause sedation in individuals who are not susceptible to sedative side effects of Generation-1 antihistaminic drugs. Likewise, a high clinical dose of diphenhydramine can be used to differentiate between patients who are susceptible to sedation and those who are not susceptible to the sedative side effects of Generation-1 antihistaminic drugs.

In one embodiment, the doctor may determine if a patient suffering from an atopic inflammatory disorder is susceptible to sedative side effects, for example, by using one of the methods described above. If the patient is susceptible to sedative side effects, the patient can safely be administered norketotifen for treatment of chronic atopic inflammatory disorders.

In one embodiment, treatment is chronic, subchronic, or acute, specifically chronic. As used herein chronic administration is three or more consecutive days of administration, specifically six or more consecutive days of administration. Acute treatment refers to a single administration. Subchronic treatment refers to less than three consecutive days of administration. In certain embodiments, chronic administration is daily administration over a period of a month, several months, to a year or more.

The embodiments disclosed herein also provide pharmaceutical compositions, which comprise the compound norketotifen, formulated together with one or more pharmaceutically acceptable carriers. The pharmaceutical compositions may be formulated for oral administration, sublingual administration, parenteral administration, dermal administration (application), transdermal administration, rectal administration, buccal administration, for topical administration, or pulmonary administration such as administration by inhalation, or insufflation of dry powder or aerosol.

Pharmaceutical compositions described herein can be administered orally, sublingually, parenterally, dermally, transdermally, rectally, buccally. The term “parenteral” administration includes intravenous, intra-arterial, intramuscular, intraperitoneal, intracutaneous, subcutaneous or intraarticular injection and infusion. The term “transdermal” includes the use of various devices (“patches” etc.) that can facilitate or modify the transport or absorption of the drug to the skin or through the skin.

Pharmaceutical compositions for oral administration of solid dosage forms include capsules, granules, pills, powders and tablets. In solid dosage forms, the active compound may be mixed with one or more pharmaceutically acceptable excipients or carriers (such as for example sodium citrate, dicalcium phosphate), fillers or extenders (such as for example starch, lactose, sucrose, glucose, mannitol, silicic acid), binders (such as for example carboxymethylcellulose, alginates, gelatin, polyvinylpyrrolidone, sucrose, acacia), humectants (such as for example glycerol), solution retarding agents (such as for example paraffin), disintegrating agents (such as for example agar-agar, calcium carbonate, starch, alginic acid, silicates, sodium carbonate), absorption accelerators (such as for example quaternary ammonium compounds), wetting agents (such as for example cetyl alcohol, glycerol monostearate), absorbents (such as for example kaolin, bentonite clay), lubricating agents (such as for example talc, calcium stearate, magnesium stearate, polyethylene glycols, sodium lauryl sulfate), and/or other excipients, such as for example buffering agents.

Solid forms of capsules, granules, pills, and tablets can have coatings and/or shells (such as for example enteric coatings) known in the art. The compositions may also be designed to release the active ingredient(s) in a certain part of the gastrointestinal tract or in a controlled release, slow-release or in a delayed-release manner. The active compound(s) can also be micro-encapsulated with one or more of the above-mentioned excipients or other suitable excipients.

Liquid dosage forms for oral administration include pharmaceutically acceptable emulsions, solutions, suspensions, syrups and elixirs. The liquid dosage form may also contain excipients known to those skilled in the art of drug formulations, such as for example diluents (such as for example water, other solvents and solubilizing agents, and mixtures thereof), and emulsifiers (such as for example ethanol, isopropyl alcohol, ethyl carbonate, ethyl acetate, benzyl alcohol, benzyl benzoate, propylene glycol, butylene glycol, dimethyl formamide, oils, oleic acid, glycerol, polyethylene glycols, sorbitan fatty esters, and mixtures thereof)

The oral compositions may also include other excipients as known to those skilled in the art.

Compositions for topical administration of norketotifen to human skin include liquids, creams, gels, suspensions, droplets, sprays, ointments and powders as well as specific delivery systems such as for examples patches, bandages. In addition to the active compound, the dermal composition may also contain other excipients as known to those skilled in the art. Creams or gels or solutions may contain 10 mg/ml to 100 mg/ml of norketotifen, calculated as free base of norketotifen but administered either as a salt or as the free base, and applied once or more times daily to the affected areas.

Pharmaceutical compositions for parenteral injections include pharmaceutically acceptable sterile aqueous or nonaqueous solutions, dispersions, suspensions, emulsions and sterile powders for reconstitution into sterile injectable solutions or dispersions prior to use. Various aqueous and nonaqueous carriers, diluents, solvents and vehicles may be used (such as for example water, ethanol, glycerol, glycol), as well as vegetable oils (such as for example olive oil), and organic esters (such as for example ethyl oleate), or mixtures of various excipients may be used. Fluidity can be maintained by use of coating material such as for example lecithin, by restricting particle size and by use of surfactants.

Parenteral compositions may also contain excipients such as preservatives, wetting agents, emulsifying agents, dispersing agents, antibacterial agents, antifungal agents, isotonic agents, and/or absorption-delaying agents. Absorption-prolonging or absorption-slowing effects may be achieved by injecting a crystalline or amorphous suspension with low water solubility. Delayed absorption may also be obtained by dissolving or suspending the drug in an oil vehicle or by using injectable depot forms (ex. microencapsulated matrices of the drug in biodegradable polymers, such as polylactide-polyglycolide, polyorthoesters, polyanhydrides) or by using various types of liposomes or microemulsions to hold the drug. Formulations for injection can be sterilized by various methods.

Pulmonary administration, such as by inhalation or insufflation, may be accomplished, for example, using an aerosolizer, a nebulizer, a dry powder inhaler, a metered dose inhaler, and the like.

The actual dosage levels of active ingredients in the pharmaceutical compositions disclosed herein may be varied so as to obtain the desired therapeutic effect. Thus the amount of drug used and the frequency of dosing varies and will depend on factors such as the administration form, the severity of the disease and other circumstances, such as for example the general health, age, and weight of the individual patient.

In general, the therapeutically active oral doses of norketotifen, useful for treating patients with the previously defined dermal, pulmonary or gastrointestinal diseases, are 0.5 mg to 20 mg orally once or multiple times daily. A preferred treatment of norketotifen is 2 mg to 10 mg, administered orally, once daily to human patients. Norketotifen may be administered orally as the free base or as a salt, such as for example a hydrochloride or a hydrogen fumarate salt.

Topical treatment with norketotifen may be in the form of for example creams or gels containing 10 mg/ml to 100 mg/ml of a salt of norketotifen or of norketotifen free base, applied one or more times daily.

Pulmonary administration of norketotifen can comprise inhalation or insufflation of aerosol formulations or dry powder formulations.

Combinations of norketotifen, administered orally to patients with dermal disorders together with norketotifen administered topically to affected areas of the skin will be useful. The oral doses of norketotifen will be 0.5 mg to 20 mg orally once or multiple times daily. The topical doses will be applied as a cream, a gel or a solution, containing norketotifen in concentrations of approximately 10 mg/ml to 100 mg/ml of the free base, but administered as the free base or as a pharmaceutically acceptable salt, and together with at least one pharmaceutically acceptable excipient for topical administration. The total dose of the dermally applied formulation of norketotifen will depend on the size of skin being treated.

In addition to the use of norketotifen as single-drug medication, embodiments disclosed herein also provide methods for co-administration of norketotifen, with at least one drug of the following classes: adrenergic antagonists, adrenergic agonists, antibacterial agents, antiviral agents, steroids, cyclooxygenase inhibitors, leukotriene antagonists, lipoxygenase inhibitors, inhibitors of specific one or more cytokines and immunosuppressants (also called immunomodulators). In specific embodiments, the norketotifen used in combination therapy together with another drug is in an amount of 0.5 mg to 20 mg orally once or multiple times daily.

When used for a dermal indication, such as for example atopic dermatitis, oral administration of norketotifen can be combined with topical application of therapeutically active doses of steroids. Exemplary steroids include a highly potent Class-I steroid such as, for example, the topical steroid clobetasol propionate cream 0.05%; a Class-II topical steroid, such as, for example, desoximethasone cream 0.025%; or a Class-III topical steroid, such as, for example, triamcinolone acetonide 0.5%. The volumes of the steroids will depend on the size of the dermal area being treated. These doses of steroids are herein referred to as therapeutically active doses. During co-administration with norketotifen, it will be possible to decrease the exposure of the patient to steroids, for example by substituting a potent steroid with a less potent steroid, such as for example replacing a Class-I steroid with a less potent Class-II steroid. A steroid-sparing effect can also be achieved by switching to a lower concentration of a particular steroid, application of a decreased amount of a steroid, or using a steroid less frequently. Such doses are reduced compared to a manufacturer's recommended dose. In one embodiment, the dose of steroid is reduced by 50% when co-administered with norketotifen. Due to the serious adverse effects of steroids, it is an advantage to the patient to use a steroid-sparing medication.

When used for dermal indication, such as for example atopic dermatitis, oral administration of norketotifen can also be combined with either oral or topical administration of an immunosuppressant drug such as for example cyclosporine, pimecrolimus or tacrolimus. A reduction of the dose of the immunosuppressant drugs is made possible with concomitant administration of norketotifen and is advantageous to the patient due to the well-known and serious side effects of immunosuppressant drugs. Such reductions of doses of immunosuppressants can be achieved by reducing the daily dose of the immunosuppressant drug or by administering the immunosuppressant less frequently than once daily. Such doses are reduced compared to a manufacturer's recommended dose. In one embodiment, the dose of immunosuppressant drug is reduced by 50% when co-administered with norketotifen. Due to the serious adverse effects of immunosuppressant drugs, it is an advantage to the patient to reduce the use of immunosuppressant drugs medication.

When used for pulmonary indications, such as for example asthma, COPD and chronic bronchitis, norketotifen can be combined with an adrenergic beta-receptor agonist and one or both of the drugs can be administered by inhalation, nasal, parenteral, topical, transdermal, rectal, sublingual or oral administration. The adrenergic beta-receptor agonist can be selected from the group consisting of albuterol (called salbutamol outside of the United States), terbutaline, fenoterol, formoterol, and salmeterol and the active isomers of the beta-receptor agonists. Since bronchial inflammation and broncoconstriction are hallmarks of asthma, it is obvious to those skilled in the art that co-administrations of the anti-inflammatory drug norketotifen and a bronchodilating adrenergic beta-agonist will be advantageous to patients suffering from asthma and other pulmonary disorders. An additional advantage is the known inhibition of beta-receptor downregulation by benzocycloheptathiophene compounds. In one embodiment, the method further comprises co-adminisering a steroid with norketotifen and an adrenergic beta-agonist.

In one embodiment, norketotifen and adrenergic beta-receptor agonist are co-administered in a formulation suitable for pulmonary administration.

The invention is further illustrated by the following non-limiting examples.

### EXAMPLES

### Example 1

### Antihistaminic Activity In Vitro

Histamine H₁ receptor binding studies were conducted utilizing human recombinant receptors. In the studies shown here, affinities of the test compounds for histamine H₁-receptors were assessed using a binding assay, where [³H-] pyrilamine was used as the ligand and the test compounds were used at increasing concentrations. The specific binding of the radioactive ligand to the receptor was defined as the difference between total binding and nonspecific binding, determined in the presence of excess unlabeled ligand. IC₅₀ values (the concentration that inhibits 50% of specific binding of the ligand) are determined by non-linear regression analysis of the competition curves.

TABLE 1
Antihistaminic activity in vitro

| ANTIHISTAMINE | H-1/IC50 (nM) |
|---|---|
| KETOTIFEN | 2.3 |
| NORKETOTIFEN | 11 |
| LORATADINE (Claritin ®) | 1,500 |
| DESLORATADINE (Clarinex ®) | 16 |
| DIPHENHYDRAMINE (Benadryl ®) | 84 |

Ketotifen is probably the most potent antihistaminic compound ever to be approved as a drug for human use. Norketotifen has less affinity for the human histamine-1 receptors than ketotifen, but is more potent, or slightly more potent, than the three reference compounds. Without being held to theory, it is believed that loratadine is a poorly active prodrug and is metabolized in the liver to desloratadine.

### Example 2

### Antihistaminic activity In Vivo

Male rats (150-200 g) were fasted overnight and twelve hours after dorsal depilation, the animals were orally pretreated with the test compound(s). Four dorsal test areas were marked with permanent ink, carefully avoiding the area closest to the spine. Sixty minutes after the dosing with the test compound, two intradermal injections of histamine (50 μl; 1.0 of histamine di-HCl) were performed, one on each side on the back of the animal. Two intradermal injections of the vehicle for the histamine solution were also performed. Evans blue dye (20 mg/kg) was injected intravenously one minute prior to the intradermal injections of histamine and the histamine vehicle. Twenty minutes were allowed for the wheal response to fully develop, whereupon the animals were euthanized and the dorsal skin with the intradermal wheals was deflected. The blue spotted areas (called “Histamine Areas”) were measured in square millimeters; the “Saline Areas” were deducted to obtain the “Histamine Effects”. In vehicle-treated animals, the Histamine Effects were on average, 94 and 82 mm² for the vehicles used during the norketotifen and ketotifen experiments, respectively. The inhibition was calculated in percent of the vehicle Histamine Effects. The results are shown in Table 2.

TABLE 2
Antihistaminic activity in vivo ( ±S.E.M.)

| Test | | | | |
|---|---|---|---|---|
| compound | Histamine | Saline | Histamine | |
| Dose | Area | Area | Effect | Inhibition |
| (mg/kg) | (mm²) | (mm²) | (mm²) | (%) |
| Vehicle* | 116 ± 5 | 22 ± 1 | 94 | — |
| Vehicle** | 107 ± 4 | 25 ± 1 | 82 | — |
| Ketotifen; 1.0 | 68 ± 6 | 21 ± 2 | 47 | 43 |
| Ketotifen; 10 | 24 ± 2 | 22 ± 3 | 2 | 98 |
| Norketotifen; 1.0 | 114 ± 8 | 22 ± 1 | 92 | 2 |
| Norketotifen; 10 | 39 ± 2 | 22 ± 1 | 17 | 82 |
| Norketotifen; 50 | 10 ± 1 | 12 ± 1 | 0 | 100 |
| DPH; 10*** | | | | 31 |

*Vehicle for norketotifen expts
**Vehicle for ketotifen expts
***DPH = diphenhydramine (Benadryl ®)

When plotted, ketotifen was found to be 2 to 3 times more potent than norketotifen as an antihistamine in these in vivo studies. Norketotifen was significantly more potent than diphenhydramine.

### Example 3

### Anti-Inflammatory Effects In Vitro

In these studies, histamine was the marker compound for inflammatory mediators that are released from mast cells and other pro-inflammatory cells in patients with atopic inflammatory diseases. The inhibition of stimulated histamine release from human leukocytes (buffy coat) by test articles was studied. The method is a modification of methods described in the prior art. Leukocytes were obtained from healthy volunteers and histamine release was induced by incubation (20 min/37° C.) with the calcium ionophore A23187 (5 μM) in the presence or absence of a test article. Histamine was analyzed by enzyme-immune assays, using commercially available kits and a microplate reader (MRX, Dynatech). The test articles were evaluated, in duplicate, at five concentrations. The results are shown in Table 3.

TABLE 3
Inhibition of inflammatory
mediator (histamine)
release; IC50 (μM)

| Test article | IC50 (μM) |
|---|---|
| Ketotifen | 91 |
| Norketotifen | 9.2 |

Norketotifen was approximately 10 times more potent than ketotifen as an inhibitor of histamine release from human pro-inflammatory cells.

### Example 4

### Anti-Inflammatory Effects In Vivo After Topical Drug Administration

In order to investigate the effects of the test compounds in dermal inflammation, a croton oil model was used. About 10 mg of a cream containing 1.0% norketotifen or a vehicle cream was applied to both ears of mice for 30 minutes. The cream was then removed and a solution of 1% croton oil in 20 μl acetone was applied to both ears. After the acetone had dried (10 seconds), the cream containing the test article (or vehicle) was reapplied and the animals were returned to their cages. At 0, 30, 60 and 90 minutes following the croton oil administration, groups of four animals were anesthetized with halothane and euthanized. Cream was wiped from the ears and ears were removed and weighed.

The effects of 10 mg of a cream containing 1.0 percent RS-norketotifen at 30, 60 and 90 minutes after administration of test articles are shown in the following table 4. All results represent mean ear weights (±S.E.M.) from 8 ears.

TABLE 4
Anti-inflammatory effects in vivo

| Time after Croton Oil | Average Ear Weight (mg) ± S.E.M | |
|---|---|---|
| Application (min) | Vehicle | RS-Norketotifen |
| 0 | 35 ± 1 | 36 ± 1 |
| (predose) | | |
| 30 | 41 ± 1 | 36 ± 1 |
| 60 | 41 ± 1 | 37 ± 2 |
| 90 | 46 ± 2 | 36 ± 2 |

In this study, the test article (racemic norketotifen) was applied topically to the ears of mice. There were obvious and potent anti-inflammatory effects after dermal application of a cream containing 1.0 percent (w/w) norketotifen.

### Example 5

### Sedative Effects in Mice

The sedation study in mice has previously been used by Schering in the loratadine project (U.S. Pat. No. 4,659,716, 1987) and by Sepracor in the desloratadine project (U.S. Pat. No. 5,595,997), which patents are hereby included by reference for their disclosure of sedation studies. In short, physostigmine (1.0 mg/kg to 2.0 mg/kg, s.c.) generally results in 100% lethality when given to groups of mice (10 mice /group) transferred into a small volume of space. Mice administered a sedating drug prior to the physostigmine injection are protected from the stress and survive. In the present study, test compounds were given orally 60 minutes prior to physostigmine injection. The number of surviving (sedated) mice was counted 30 minutes after injection of the physostigmine dose. Results are shown from tests that were performed between the years 1997 and 2009 in Table 5.

TABLE 5
Sedative effects in mice

| | Oral dose | Sedated |
|---|---|---|
| | (mg/kg) | animals |
| VEHICLE | — | 0/10 |
| NORKETOTIFEN | 83 | 3/10 |
| NORKETOTIFEN | 100 | 3/10 |
| NORKETOTIFEN | 150 | 3/10 |
| NORKETOTIFEN | 180 | 6/10 |
| S-NORKETOTIFEN | 100 | 0/10 |
| S-NORKETOTIFEN | 150 | 0/10 |
| R-NORKETOTIFEN | 100 | 3/10 |
| R-NORKETOTIFEN | 150 | 3/10 |
| KETOTIFEN (Zaditen ®; Gen-1) | 25 | 5/10 |
| KETOTIFEN (Zaditen ®; Gen-1) | 50 | 8/10 |
| KETOTIFEN (Zaditen ®; Gen-1) | 100 | 10/10 |
| CYPROHEPTADINE (Periactin ®; Gen-1) | 100 | 9/10 |
| PYRILAMINE (Mepyramine ®; Gen-1) | 100 | 8/10 |
| HYDROXYZINE (Atarax ®; Gen-1) | 100 | 9/10 |
| DIPHENHYDRAMINE (Benadryl ®; Gen-1) | 50 | 5/10 |
| DIPHENHYDRAMINE (Benadryl ®; Gen-1) | 100 | 8/10 |
| ASTEMIZOLE (Hismanal ®; Gen-2) | 100 | 1/10 |
| NORASTEMIZOLE (Soltara ™; Gen-2) | 100 | 0/10 |
| LORATADINE (Claritin ®; Gen-2) | 150 | 1/10 |
| DESLORATADINE (Clarinex ®; Gen-2) | 150 | 0/10 |
| TERFENADINE (Seldane ®; Gen-2) | 150 | 0/10 |
| FEXOFENADINE (Allegra ®; Gen-2) | 150 | 0/10 |

Gen-1 = Generation-1 (sedating antihistamines)
Gen-2 = Generation-2 (non-sedating antihistamines)

All registered Generation-1 antihistamines were sedating in mice and all registered Generation-2 antihistamines were free from sedation, using the physostigmine lethality test. Racemic norketotifen expressed sedative activity in this test system.

### Example 6

### Sedative Effects in Humans

Sedation studies were performed in human volunteers, where the volunteers were administered norketotifen orally at doses of 5 mg or 10 mg (o.d. and b.i.d.), which is believed to be about a 10-fold and 20-fold, respectively, higher dose than the amount of norketotifen formed as a metabolite after a clinical dose of ketotifen 1.0 mg. The test articles were administered in gelatin capsules and vehicle-capsules were empty. The volunteers were dosed at 8-10 AM and had been fasted overnight before dosing. All observations regarding sedation/sleepiness were made by the individual volunteers and by an experienced scientist with previous experience in CNS-studies. As a positive control ketotifen was administered in an oral dose of 2 mg to the volunteers (two tablets Zaditen®, Paladin, each tablet containing 1 mg ketotifen). The test articles were in the form of hydrogen fumarate salts.

Humans are known to express sedation within one hour after a single oral dose of Generation-1 antihistamines, such as for example diphenhydramine (Benadryl®, McNeill) or ketotifen (Zaditen®, Novartis). The test article was therefore usually administered for one day only, although some experiments were performed with the test article being administered for three consecutive days.

The test results demonstrated complete lack of sedation in all volunteers who had been administered racemic norketotifen at oral doses of 5 mg or 10 mg. The test results also demonstrated that ketotifen caused sedation in the human volunteers, which is not surprising, since sedation/drowsiness is a dose-dependent and dose-limiting side effect of ketotifen.

Surprisingly, norketotifen did not cause sedation in the human volunteers, which is contrary to the results from earlier animal studies using the mouse physostigmine model, which had been considered to be of high predictive value. To our knowledge, this is the first study that has been performed to specifically study sedative side effects of RS-norketotifen in humans and it has now surprisingly been found that racemic norketotifen is completely free from sedative effects in humans, even after administration of high oral doses.

### Example 7

### Toxicological Effects

Acute toxicological studies were performed in rats (Sprague-Dawley; M and F; 200-250 grams). The animal-sparing Up-and-Down Procedure (FDA, OECD) was used. Both oral and intravenous toxicity tests were performed.

The acute toxicity, expressed as estimated LD50 and calculated in mg/kg body weight of norketotifen and ketotifen are shown in Table 7. Both compounds were administered as hydrogen fumarate salts.

TABLE 7
Toxicological effects

| | Acute toxicity | |
|---|---|---|
| | (estimated LD50) mg/kg | |
| TEST SYSTEM | NORKETOTIFEN | KETOTIFEN |
| RAT; intravenous | 10-15 | 5-10 |
| RAT; oral | 1500-2000 | <300 |

Norketotifen is significantly less toxic than ketotifen after intravenous or oral administration.

### Example 8

### Exemplary Oral Dosage Formulation

TABLE 8
Tablet formulations

| | Amount | | Amount | |
|---|---|---|---|---|
| | per | | per | |
| Ingredient | tablet | | batch | |
| Norketotifen | 4 | mg | 40 | g |
| Microcrystalline cellulose | 30 | mg | 300 | g |
| Lactose | 70 | mg | 700 | g |
| Calcium stearate | 2 | mg | 20 | g |
| FD&C Blue #1 Lake | 0.03 | mg | 300 | mg |

The active ingredient is blended with the lactose and cellulose until a uniform blend is formed. The blue lake is added and further blended. Finally, the calcium stearate is blended in, and the resulting mixture is compressed into tablets using for example a 9/32-inch (7 mm) shallow concave punch. Tablets of other strengths may be prepared by altering the ratio of active ingredient to the excipients or to the final weight of the tablet.

The terms “pharmaceutically acceptable salts” or “a pharmaceutically acceptable salt thereof” refer to norketotifen salts, which have been prepared from pharmaceutically acceptable non-toxic acids. Suitable pharmaceutically acceptable acid addition salts for the compound described herein include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, p-toluenesulfonic, succinic, sulfuric, tartaric, and the like. The hydrogen fumarate salt and the hydrochloride salt are particularly preferred.

Those skilled in the art will recognize, or be able to ascertain, using no more than routine experimentation, many equivalents to the specific embodiments described herein.

Such equivalents include co-administration of the active compound with any other drug that is used to treat diseases, mentioned in this document.

Other equivalents include numerous pharmaceutically acceptable salt forms e.g. sulfate, hydrobromide, hydrochloride, dihydrochloride, methanesulphonate, fumarate, hydroxynaphthoate or where appropriate one or other of the hydrate forms thereof. Such equivalents also include co-administration of the active compound with any other drug that is used to treat the diseases, mentioned herein.

Those skilled in the art of medicine will realize that higher or lower doses than those indicated here may be used or preferred and the doses may be given more or less frequently than suggested here.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A method for treating a chronic inflammatory dermal disorder in a human patient in need thereof, comprising orally administering to the human patient a therapeutically effective amount of 0.5 to 20 mg of RS-norketotifen or a pharmaceutically acceptable salt thereof dosed once or more daily, wherein administration is three or more consecutive days of administration, wherein the therapeutically effective amount of norketotifen or a pharmaceutically acceptable salt thereof does not produce sedative side effects in the human patient, and wherein the amount of said RS-norketotifen is calculated as free base.

2. The method of claim 1, wherein the human patient is susceptible to sedative side effects upon administration of Generation-1 antihistamines.

3. The method of claim 1, wherein said dermal disorder selected from the group consisting of eczema, atopic dermatitis, urticaria and psoriasis.

4. The method of claim 1, further comprising co-administering a therapeutically active dose of a steroid.

5. The method of claim 4, wherein the steroid is topically administered.

6. The method of claim 1, further comprising co-administering a reduced dose of a steroid compared to a manufacturer's recommended dose of the steroid.

7. The method of claim 1, further comprising co-administering a reduced dose of an immunosuppressant compared to a manufacturer's recommended dose of the immunosuppressant.
